(19) United States
(12) Plant Patent Application Publication
(10) Pub. No.: US 2023/0301221 P1
(43) Pub. Date: Sep. 21, 2023

(54) Cannabis plant named 'DRG3'

(71) Applicant: Michael Ray Fowler, McKinleyville, CA (US)

(72) Inventor: Michael Ray Fowler, McKinleyville, CA (US)

(21) Appl. No.: 18/445,138

(22) Filed: Apr. 21, 2023

Related U.S. Application Data
(63) Continuation-in-part of application No. 17/456,133, filed on Nov. 22, 2021.
(60) Provisional application No. 63/117,135, filed on Nov. 23, 2020.

(51) Int. Cl. A01H 6/28

(52) U.S. Cl. PLT/258

(57) ABSTRACT

The unique annual herbaceous cannabis plant variety ‘DRG3’ is provided. The variety can be distinguished by its outstanding features of a distinct “oranges and spice” flavor profile and a distinct purple subset leaf and flower colors.

### CROSS REFERENCE TO RELATED APPLICATIONS

The present Application for Patent is a continuation-in-part of U.S. Application No. 17/456,133 filed on Nov. 22, 2021, which in turn claims the benefit of provisional application No. 63/117,135 entitled “CANNABIS PLANT NAMED ‘DRG3 ’” filed Nov. 23, 2020, and hereby expressly incorporated by reference herein.

Latin name of the genus and species: Genus - Cannabis. Species - sativa.

Variety denomination: The new cannabis plant claimed is of the variety denominated ‘DRG3’.

### BACKGROUND OF THE INVENTION

### Field of the Invention

The present invention relates to a new and distinct annual variety of C. sativa, which has been given the variety denomination of ‘DRG3’. ‘DRG3’ is intended for use as use as a medicinal herb for sale in Cannabis dispensaries.

### Background of the Related Art

The genus Cannabis has been in use by humans for millennia, due to the multiplicity of its benefits to humans, including the considerable value and utility of its fiber, the nutritional value of its seeds, and the medicinal value of its floral parts and products made from them. Currently the genus is under intense legal commercialization in the United States as industrial hemp for a variety of purposes including biodegradable plastics and building materials, clothing, paper, food, fuel and medicines.

Cannabidiol (CBD) extracted from Cannabis is widely used in over-the-counter medicines and topical treatments, and is also the active ingredient in the FDA-approved drug Epidiolex. CBD is just one of at least dozens-perhaps hundreds--of cannabinoids endogenous to Cannabis, tetrahydrocannabinol (THC) being the other cannabinoid that is most well-known. The cannabinoids as a group interact with the human endocannabinoid receptors, which are distributed in the brain and throughout the body. The study of the endocannabinoid system (ECS) in humans and other mammals is an area of increasing interest and holds tremendous promise for the future of medicine. See, e.g., Russo (2019). Cannabis and Pain, Pain Medicine, 20(10): 1093/pm/pnz227; and Russo (2016). Clinical Endocannabinoid Deficiency Reconsidered: Current Research Supports the Theory in Migraine, Fibromyalgia, Irritable Bowel, and Other Treatment-Resistant Syndromes, Cannabis Cannabinoid Res. 1(1): 154-165.

Typically, marijuana products are available to users for purchase in specialized “dispensaries” that offer dried flower, edibles, tinctures, extracts, and the like. In some cases, a unique or unusual chemical profile, or chemotype, is attractive not only for flower sales but also for use in the preparation of extracts and/or isolates and for the manufacture of a variety of products that possess characteristics of the chemotype.

### SUMMARY OF THE INVENTION

The present invention relates to a new and distinct annual variety of C. sativa, which has been given the variety denomination of ‘DRG3’. ‘DRG3’ is intended for use as use as a medicinal herb for sale in Cannabis dispensaries.

The new C. sativa variety is a selection from pollen breeding of parent ‘Orange Cream’ (not patented) with parent ‘Razzleberry Kush’ (not patented). The new variety was discovered and selected as a single plant within a population of 250 resulting C. sativa plants from this controlled pollination in 2015 in a controlled greenhouse at Humboldt County, CA. Selection criteria were isolation of a superior female with genetic traits of both parents, excellent visual presentation and desired medicinal effects upon consumption.

Asexual reproduction of the new variety by cutting propagation since 2016 at Humboldt County, CA has demonstrated that the new variety reproduces true to type with all of the characteristics, as herein described, firmly fixed and retained through successive generations of such asexual propagation.

The following characteristics of the new variety have been repeatedly observed and can be used to distinguish ‘DRG3’ as a new and distinct variety of C. sativa plant:

1. Full “purple” leaf color saturation to a “near black” in outdoor-grown methodologies.

2. Distinct “Smoked Oranges” flavor profile upon combustion.

3. Fast flowering time - indoors; under 60 days, outdoors; mid-late September.

4. Short, compact, and very dense flower buds with “purple” bracts and “golden” stigmas.

Plants of the new variety differ from plants of the seed parent ‘Orange Cream’ in overall plant structure, leaf and flower colors, bud density, and flower aroma. Plants of the new variety differ from plants of the pollen parent ‘Razzleberry Kush’ primarily in branch and plant structure, plant height, and flower flavor profile. ‘DRG3’ has an “orange” or citrus essence in its flavor profile, while combining desirable attributes of both parents in plant color, size, and flower development with high therapeutic value for its anxiety-reduction and stress-reduction qualities and pain-relief values.

‘DRG3’ can have a cannabinoid profile as set forth in Table 1 and a terpene profile as set forth in Table 2. The chemotypic characteristics of the new variety are variable based upon cultivation conditions, as is typical of Cannabis plants.

TABLE 1

Exemplary Profiles of Key Cannabinoids

| | Percent | Percent | Percent |
|---|---|---|---|
| Total Cannabinoids*** | 18.2728% | 22.841% | 27.4092% |
| Total THC* | 17.6632% | 22.079% | 26.4948% |
| THCa | 18.7448% | 23.431% | 28.1172% |
| Δ9THC | 1.2240% | 1.530% | 1.8360% |
| CBCa | 0.2720% | 0.340% | 0.4080% |
| CBGa | 0.1920% | 0.240% | 0.2880% |
| CBDa | 0.1160% | 0.145% | 0.1740% |
| CBG | 0.0720% | 0.090% | 0.1080% |
| THCVa | 0.0320% | 0.040% | 0.0480% |
| Sum of Cannabinoids | 20.6528% | 25.816% | 30.9792% |

^{∗}Total THC = Δ9THC + (THCa * 0.877)
^{∗∗}Total CBD = CBD + (CBDa * 0.877)
^{∗∗∗}Total Cannabinoids = Total THC + Total CBD +Total CBG + Total THCV + Total CBC +Total CBDV + Δ8THC + CBL + CBN

TABLE 2

Exemplary Profiles of Key Terpenes

| | Percent | Percent | Percent |
|---|---|---|---|
| Beta Caryophyllene | 0.878% | 1.097% | 1.316% |
| Limonene | 0.304% | 0.380% | 0.456% |
| Alpha Humulene | 0.278% | 0.348% | 0.418% |
| Myrcene | 0.136% | 0.170% | 0.204% |
| Alpha Bisabolol | 0.112% | 0.140% | 0.168% |
| Guaiol | 0.106% | 0.132% | 0.158% |
| Linalool | 0.096% | 0.120% | 0.144% |
| Beta Pinene | 0.048% | 0.060% | 0.072% |
| Terpineol | 0.032% | 0.040% | 0.048% |
| Alpha Pinene | 0.026% | 0.032% | 0.038% |
| Fenchol | 0.024% | 0.030% | 0.036% |
| Caryophyllene Oxide | 0.024% | 0.030% | 0.036% |
| Nerolidol | 0.012% | 0.015% | 0.018% |
| Valencene | 0.006% | 0.007% | 0.008% |
| Total Terpenoids | 2.081% | 2.601% | 3.121% |

### BRIEF DESCRIPTION OF THE DRAWINGS

The accompanying photographic illustrations show the typical appearance of the new variety ‘DRG3’. The colors are as nearly true as is reasonably possible in a color representation of this type. Colors in the photographs may differ slightly from the color values cited in the detailed botanical description which accurately describes the colors of the new plant.

FIG. 1 is a photograph of the flower and leaves of the new variety ‘DRG3’.

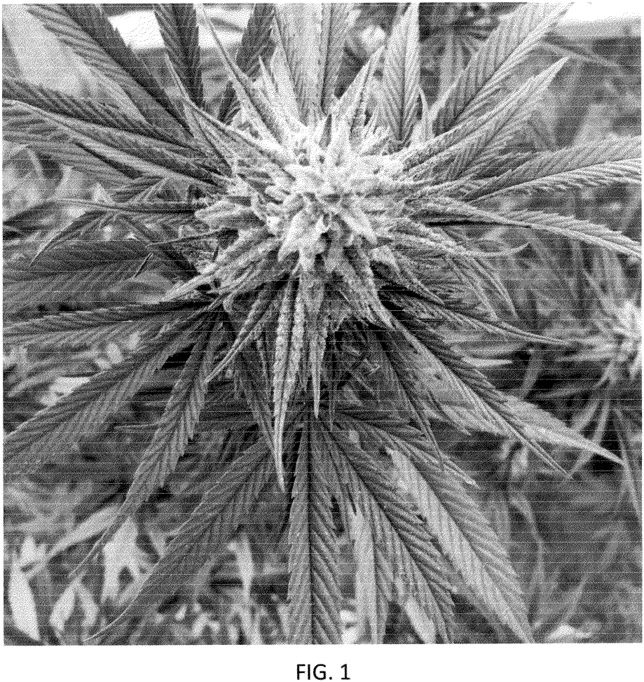

FIG. 2 is a photograph of the flower and leaves of the new variety ‘DRG3’.

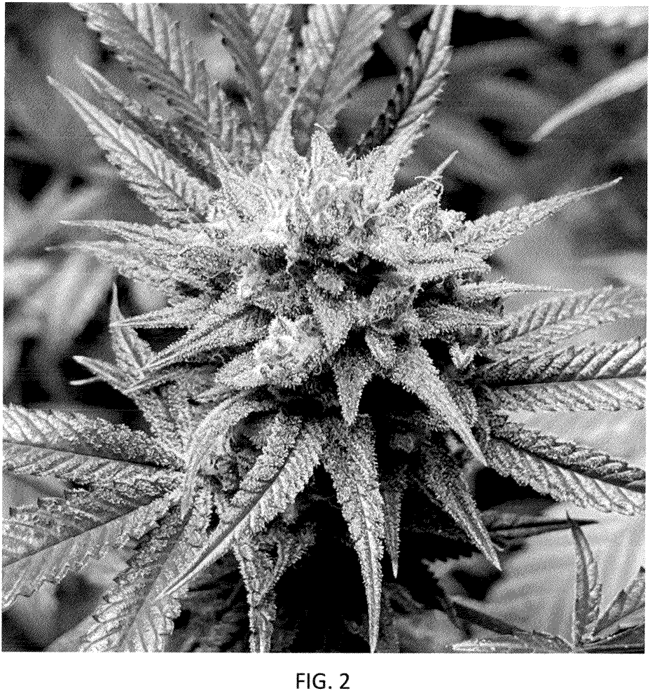

FIG. 3 is a photograph of the flower and leaves of the new variety ‘DRG3’.

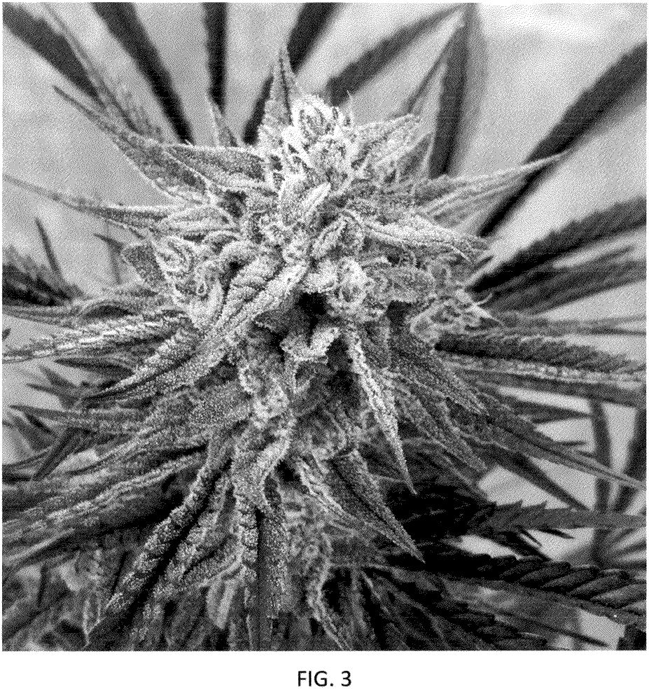

FIG. 4 is a photograph of the flower and leaves of the new variety ‘DRG3’.

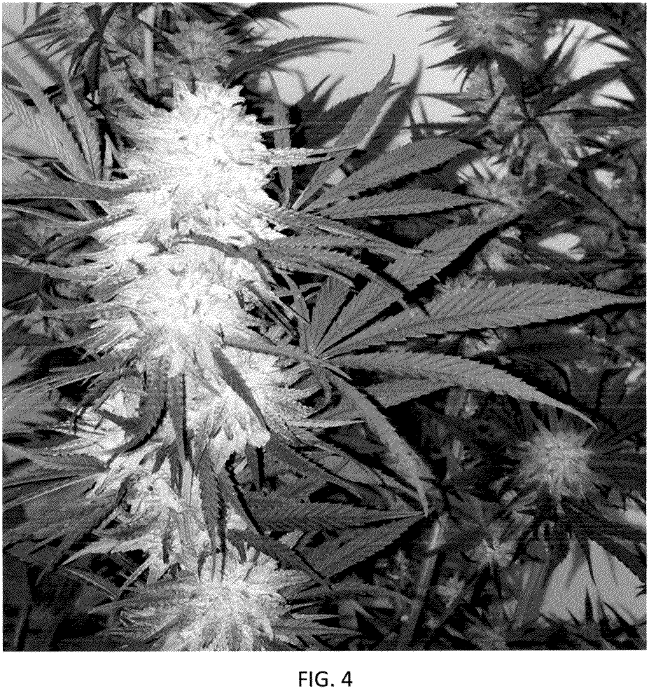

### DETAILED BOTANICAL DESCRIPTION

The following detailed description sets forth the distinctive characteristics of ‘DRG3’. The data which defines these characteristics were collected from asexual reproductions of the original selection. Dimensions, sizes, colors, and other characteristics are approximations and averages set forth as accurately as possible. The plant history was taken on plants approximately 1-3 years of age, and the descriptions relate to plants grown in Mendocino and Humboldt County, CA.
Type: Herbaceous tap-rooted annual.
Classification:
   a. Family.—Cannabaceae.
   b. Genus.—Cannabis.
   c. Species.—sativa.
   d. Common Name.—Marijuana.
Plant:
   General.—a. Origin — Humboldt County, CA. b. Parentage: a. Male parent - Razzleberry Kush. b. Female parent - Orange Cream. c. Growth habit — Vigorous and compact. d. Height — 40′-60″. e. Plant spread — 30″-50″. f. Growth rate — Very fast with quick flower set. g. Branching characteristics — Tight, compact and upswept. h. Length of primary lateral branches — 36′-48″. i. Quantity of primary lateral branches — > 12. j. Characteristics of primary lateral branches: a. Color — Green with purple veining. b. Texture — Ribbed and rough. c. Strength — Moderate to hardy. .k. Internode length — 2″-4″ (4-12 cm). l. Cold hardiness — Semi-hardy. m. Cold tolerance — >40. n. Shipping tolerance — >50 <80.
Foliage:
   General: A short, compact, and wide canopy of close set flower nodes with an indica leaning appearance.. a. Leaf — 5-7 Defined Fingers of medium size. b. Arrangement — Opposite descussate with switch to alternate in flower. c. Quantity — Varies by height and pinch. d. Leaf color (Top side) — Medium green. e. Leaf color (Under side) — Light green. f. Leaf arrangement — Opp. dec. to alternate in flowering. g. Leaf shape — 5-7 Finger Sessile Leaflets. h. Leaf margins — Serrate. i. Undulation of margin — Typical to species and varies by leaf size. j. Leaf apices — Pointed attenuate. k. Leaf bases — Sagittate. l. Leaf width — 9-17 cm. m. Leaf length — 9-19 cm. n. Texture of top and bottom surfaces —Matte veined top and bottom. o. Appearance of top and bottom surfaces — Smooth medium green and matte light green sheen. p. Venation type — Palmate. q. Petiole length — 4.5-8 cm. r. Petiole color — Maroon top half and light green bottom half. s. Petiole texture — Bi-color ribbed and fuzzy to touch.
Inflorescence:
   General: A Complex Pinnacle.—a. Natural flowering season — First to middle of September. b. Inflorescence and flower type and habit — A main branch Pinnacle. c. Fragrance — Oranges and Spice. d. Female flower and bud: i. Bud shape — Urn Panicle. ii. Bud length — 4.5 cm. iii. Bud diameter — 2.5 cm. iv. Bract diameter — 0.3 cm. v. Bract length — 0.4-0.6 cm. vi. Bract quantity per flower — Varies by size. vii. Bract shape — Ovate. viii. Bract base — Perigonal. ix. Bract texture — Fuzzy. x. Stigma length — 0.5-0.7 cm. xi. Stigma color — White/cream/red. xii. Stigma number — 2. xiii. Stigma shape — Round hirsute. xiv. Ovary position — Bracts base. xv. Ovary shape — Ovate. xvi. Ovary length — 1/32″+/- (0.1 cm+/-).
Seeds:
   a. Shape.—Obtuse Oval.
   b. Length.—0.4 cm.
   c. Width.—0.2 cm+/-.

1. A new and distinct variety of Cannabis plant named ‘DRG3’, as illustrated and described herein.
